(19) United States
(12) Patent Application Publication
(10) Pub. No.: US 2024/0269515 A1
(43) Pub. Date: Aug. 15, 2024

(54) LOAD SPECIFYING METHOD, LOAD SPECIFYING DEVICE, AND COMPUTER PROGRAM

(71) Applicant: SEIKO EPSON CORPORATION, Tokyo (JP)

(72) Inventors: Eiichiro YAMAGUCHI, MATSUMOTO-SHI (JP); Ryoki WATANABE, MATSUMOTO-SHI (JP)

(21) Appl. No.: 18/437,282

(22) Filed: Feb. 9, 2024

(30) Foreign Application Priority Data
Feb. 13, 2023 (JP) 2023-019818

(51) Int. Cl. A63B 24/00; A61B 5/00

(52) U.S. Cl. CPC A63B 24/0087; A61B 5/7264; A63B 2024/0093

(57) ABSTRACT

A load specifying method for a muscle strength training device includes: acquiring a known feature spectrum for each of a plurality of pieces of time-series waveform data; acquiring target time-series waveform data; acquiring a target feature spectrum for each of a plurality of pieces of target time-series waveform data; and specifying a spectrum similarity satisfying a predetermined extraction condition and specifying a candidate load corresponding to the target time-series waveform data as a calculation source of the specified spectrum similarity.

The present application is based on, and claims priority from JP Application Serial Number 2023-019818, filed Feb. 13, 2023, the disclosure of which is hereby incorporated by reference herein in its entirety.

### BACKGROUND

### 1. Technical Field

The present disclosure relates to a load specifying technique for a muscle strength training device.

### 2. Related Art

In related art, a technique has been known in which a muscle mass index value of a user is calculated based on an ultrasonic image, and guideline information on a load and the number of times of training using a muscle strength training device is generated based on the calculated muscle mass index value.

JP-A-2015-142619 is an example of the related art.

In the technique in the related art, for example, at start of the training, an extrapolation line connecting a set target value and the calculated muscle mass index value is obtained, and the load and the number of times of the training are determined along the extrapolation line. However, in the technique in the related art, the same training load may be set for a plurality of users who have the same muscle mass index value and target value at the start of the training. Since a degree of muscle growth in response to a load varies depending on the user, in the technique in the related art, it may not be possible to set appropriate loads for various users.

### SUMMARY

According to a first aspect of the present disclosure, a load specifying method for a muscle strength training device is provided. The load specifying method includes: a step (a) of preparing, for each of a plurality of subjects, time-series waveform data related to a fascia of the subject when the subject performs muscle strength training for which a reference load is set such that a muscle gain amount of the subject per unit time satisfies a predetermined condition; a step (b) of inputting a plurality of pieces of the time-series waveform data to a vector neural network-based trained machine learning model having a plurality of vector neuron layers and acquiring a known feature spectrum as a feature spectrum from output of a specific layer of the trained machine learning model for each of the plurality of pieces of time-series waveform data; a step (c) of acquiring target time-series waveform data, which is the time-series waveform data for each of different candidate loads, when a user executes a plurality of types of muscle strength training for which the different candidate loads are set; a step (d) of inputting a plurality of pieces of the target time-series waveform data to the trained machine learning model and acquiring a target feature spectrum as the feature spectrum from the output of the specific layer for each of the plurality of pieces of target time-series waveform data; and a step (e) of calculating a spectrum similarity, which is a similarity between each of a plurality of the known feature spectra and each of a plurality of the target feature spectra, specifying the spectrum similarity satisfying a predetermined extraction condition among a plurality of the calculated spectrum similarities, and specifying the candidate load corresponding to the target time-series waveform data as a calculation source of the specified spectrum similarity.

According to a second aspect of the present disclosure, a load specifying device for a muscle strength training device is provided. The load specifying device includes: a storage device configured to store a vector neural network-based trained machine learning model having a plurality of vector neuron layers; a first spectrum acquisition unit configured to, when a subject performs muscle strength training for which a reference load is set such that a muscle gain amount of the subject per unit time satisfies a predetermined condition, input time-series waveform data related to a fascia corresponding to each of the plurality of subjects to the trained machine learning model and acquire a known feature spectrum as a feature spectrum from output of a specific layer of the trained machine learning model for each of the plurality of pieces of time-series waveform data; a waveform acquisition unit configured to acquire target time-series waveform data, which is the time-series waveform data for each of different candidate loads, when a user executes a plurality of types of muscle strength training for which the different candidate loads are set; a second spectrum acquisition unit configured to input a plurality of pieces of the target time-series waveform data to the trained machine learning model and acquire a target feature spectrum as the feature spectrum from the output of the specific layer for each of the plurality of pieces of target time-series waveform data; and a specifying unit configured to calculate a spectrum similarity, which is a similarity between each of a plurality of the known feature spectra and each of a plurality of the target feature spectra, specify the spectrum similarity satisfying a predetermined extraction condition among a plurality of the calculated spectrum similarities, and specify the candidate load corresponding to the target time-series waveform data as a calculation source of the specified spectrum similarity.

According to a third aspect of the present disclosure, a non-transitory computer-readable storage medium storing a program causing a computer to execute load specification for a muscle training device is provided. The program includes: a function (a) of storing a vector neural network-based trained machine learning model having a plurality of vector neuron layers; a function (b) of, when a subject performs muscle strength training for which a reference load is set such that a muscle gain amount of the subject per unit time satisfies a predetermined condition, inputting time-series waveform data related to a fascia corresponding to each of a plurality of the subjects to the trained machine learning model and acquiring a known feature spectrum as a feature spectrum from output of a specific layer of the trained machine learning model for each of the plurality of pieces of time-series waveform data; a function (c) of acquiring target time-series waveform data, which is the time-series waveform data for each of different candidate loads, when a user executes a plurality of types of muscle strength training for which the different candidate loads are set; a function (d) of inputting a plurality of pieces of target time-series waveform data to the trained machine learning model and acquiring a target feature spectrum as the feature spectrum from the output of the specific layer for each of the plurality of pieces of target time-series waveform data; and a function (e) of calculating a spectrum similarity, which is a similarity between each of a plurality of the known feature spectra and each of a plurality of the target feature spectra, specifying the spectrum similarity satisfying a predetermined extraction condition among a plurality of the calculated spectrum similarities, and specifying the candidate load corresponding to the target time-series waveform data as a calculation source of the specified spectrum similarity.

### BRIEF DESCRIPTION OF THE DRAWINGS

FIG. 1 is a diagram showing a load specifying system according to a first embodiment.

FIG. 2 is a diagram showing a sensor device.

FIG. 3 is a diagram showing a configuration of a machine learning model.

FIG. 4 is a diagram showing another configuration of a trained machine learning model.

FIG. 5 is a diagram showing processing of generating the trained machine learning model.

FIG. 6 is a diagram showing a plurality of training sets.

FIG. 7 is a diagram showing a feature spectrum.

FIG. 8 is a diagram showing a configuration of a known feature spectrum group.

FIG. 9 is a flowchart of load specifying processing executed by the load specifying system.

FIG. 10 is a diagram showing a first calculation method of a spectrum similarity.

FIG. 11 is a diagram showing a second calculation method of the spectrum similarity.

FIG. 12 is a diagram showing a third calculation method of the spectrum similarity.

### DESCRIPTION OF EMBODIMENTS

### A. Embodiment

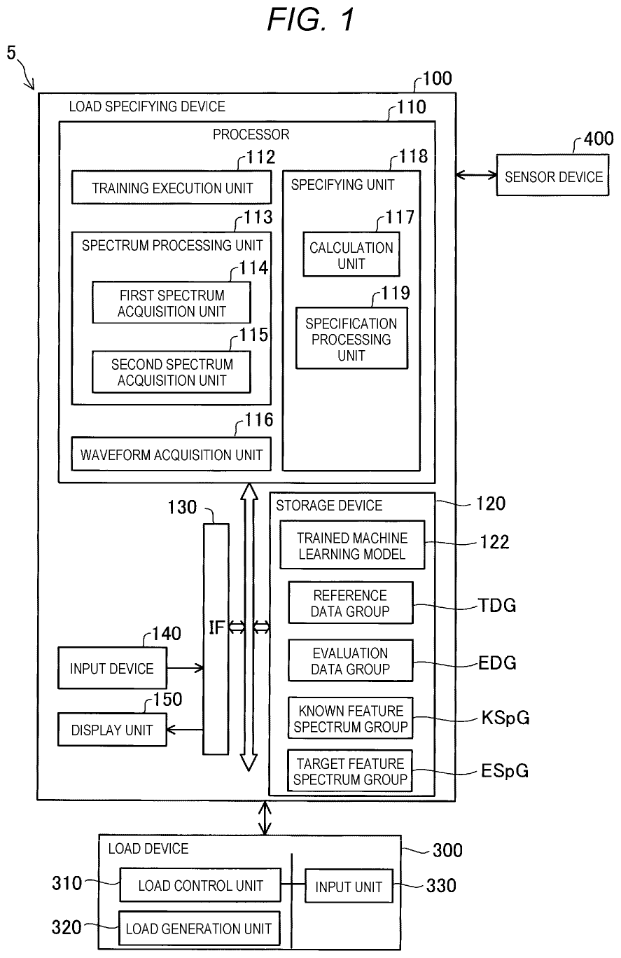

FIG. 1 is a diagram showing a load specifying system 5 according to an embodiment. The load specifying system 5 is a system that specifies an appropriate load of a muscle strength training device for a user who is a target of muscle strength training using time-series waveform data that is related to a fascia and acquired from a plurality of subjects by performing the muscle strength training with the muscle strength training device. The load specifying system 5 includes a load specifying device 100, a sensor device 400, and a load device 300 as a muscle strength training device. The load specifying device 100 and the sensor device 400, and the load specifying device 100 and the load device 300 communicate data with each other wired or wirelessly.

The load device 300 is a muscle strength training device that generates a physical load. Examples of the load device 300 include a cable machine, a treadmill, and an exercise bike (registered trademark). The load device 300 includes a load control unit 310, a load generation unit 320, and an input unit 330. The load control unit 310 controls an operation of the load device 300. For example, the load control unit 310 adjusts a load of the load generation unit 320. When the load device 300 is an electromagnetic load type exercise bike, the load control unit 310 adjusts a current flowing through an electromagnet incorporated into a rotary wheel by controlling a voltage applied to the electromagnet. Accordingly, a load of a pedal, which is the load generation unit 320, can be changed by changing easiness of rotation of the rotary wheel. The load generation unit 320 is an element that generates a load. When the load device 300 is an exercise bike, the load generation unit 320 is a pedal. The input unit 330 receives, for example, input of a load value to be set. The input unit 330 is, for example, a touch panel.

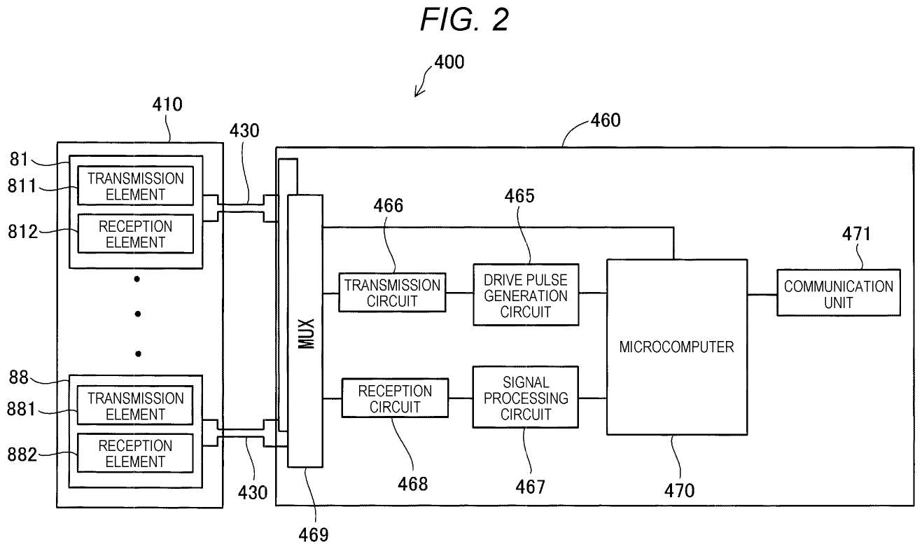

Before describing the load specifying device 100 with reference to FIG. 1, the sensor device 400 will be described in detail with reference to FIG. 2. FIG. 2 is a diagram showing the sensor device 400. The sensor device 400 is a device that acquires data related to a muscle of a person such as a subject or a user when muscle strength training is performed using the load device 300. The sensor device 400 according to the embodiment is an ultrasonic generating device, and acquires time-series waveform data related to a fascia of the person.

The sensor device 400 includes an ultrasonic probe 410 that includes an ultrasonic element for emitting and receiving ultrasonic waves, and a control unit 460 that controls functions of the ultrasonic probe 410 and performs signal processing. The ultrasonic probe 410 and the control unit 460 are electrically coupled to each other by interconnects 430.

The ultrasonic probe 410 includes eight ultrasonic elements 81 to 88. In FIG. 1, illustration of six ultrasonic elements 82 to 87 other than the ultrasonic element 81 and the ultrasonic element 88 is omitted. The ultrasonic element 81 includes an ultrasonic transmission element 811 and an ultrasonic reception element 812. The ultrasonic element 88 includes an ultrasonic transmission element 881 and an ultrasonic reception element 882. Similar to the ultrasonic element 81 and the ultrasonic element 88, the six ultrasonic elements 82 to 87 each also include an ultrasonic transmission element and an ultrasonic reception element. Here, the expression “include an ultrasonic transmission element and an ultrasonic reception element” is a functional description, and structurally, one ultrasonic element has a function of a “transmission element” and a function of a “reception element”. The eight ultrasonic elements 81 to 88 are disposed in a row at equal intervals on, for example, a flat base member. The ultrasonic elements 81 to 88 are attached, via fixing members, such as an adhesive pad and a belt, on a measurement target site for acquiring the time-series waveform data related to the fascia of the person. The number of the ultrasonic elements 81 to 88 is not limited thereto, and may be one or more.

The control unit 460 generates the time-series waveform data related to the fascia of the person based on reception signals sequentially received from the reception elements 812 to 882 of the respective ultrasonic elements 81 to 88. Ultrasonic waves transmitted from the transmission elements 811 to 881 toward the measurement target site of the person are reflected by a fascia on the measurement target site. An intensity of the reflected wave changes according to a thickness of the fascia. When the intensity of the reflected wave is received by the reception elements 812 to 882, the control unit 460 generates the time-series waveform data related to the fascia. That is, the time-series waveform data is data representing a fascia movement, which is a muscle contraction operation in the measurement target site during muscle strength training. In the embodiment, the control unit 460 generates the time-series waveform data by arranging the reception signals, which are the reflected waves received from the reception elements 812 to 882, in time series during a measurement period that is at least part of a period during which the person is performing the muscle strength training using the load device 300.

The control unit 460 includes a drive pulse generation circuit 465, a transmission circuit 466, a signal processing circuit 467, a reception circuit 468, a multiplexer 469, a microcomputer 470, and a communication unit 471. The drive pulse generation circuit 465 generates a pattern of a predetermined drive frequency and a wave number when transmitting ultrasonic waves. The transmission circuit 466 outputs a transmission waveform of a predetermined drive voltage according to the generated pattern. Each of the transmission elements 811 to 881 transmits the transmission waveform output from the transmission circuit 466 as an ultrasonic wave to the measurement target site. When receiving the reflected wave of the ultrasonic wave, each of the reception elements 812 to 882 receives the reflected wave as a reception signal. The reception circuit 468 amplifies the reception signals received by the reception elements 812 to 882. The signal processing circuit 467 performs data processing, such as envelope processing, on the reception signals amplified by the reception circuit 468 to generate the time-series waveform data. The microcomputer 470 is configured such that the multiplexer 19 sequentially switches transmission and reception operations of the ultrasonic elements 81 to 88. The microcomputer 470 transmits, via the communication unit 471, the time-series waveform data generated by the load specifying device 100. In the embodiment, the microcomputer 470 arranges eight pieces of time-series waveform data generated by receiving the reception signals from the reception elements 812 to 882 of the respective ultrasonic elements 81 to 88 in order of the reception elements 812 to 882 to generate one piece of time-series waveform data used in the load specifying device 100. In another embodiment, the microcomputer 470 may combine the eight pieces of time-series waveform data generated by receiving the reception signals from the reception elements 812 to 882 of the respective ultrasonic elements 81 to 88, and generate one piece of time-series waveform data used in the load specifying device 100 by, for example, averaging. In still another embodiment, the microcomputer 470 may use, as one piece of time-series waveform data used in the load specifying device 100, time-series waveform data generated by receiving the reception signal from one specific reception element among the eight pieces of time-series waveform data received and generated from the reception elements 812 to 882 of the respective ultrasonic elements 81 to 88.

Here, a large amount of ultrasonic waves are reflected in a fascia, which is a boundary portion between a fat layer and a muscle layer of a person, and ultrasonic waves are hardly reflected in the fat layer or the muscle layer. By utilizing the property of the ultrasonic waves, in a depth direction from above the measurement target site where the ultrasonic probe 410 is disposed toward the measurement target site, respective positions of one fascia, which is a boundary portion where one side of the muscle layer is in contact with the fat layer, and the other fascia, which is a boundary portion where the other side of the muscle layer is in contact with the fat layer, can be obtained by the reflected waves. The microcomputer 470 of the sensor device 400 can calculate a distance between the one fascia and the other fascia as a thickness of the muscle layer. Since the thickness of the muscle layer has a positive correlation with a muscle mass, the thickness of the muscle layer is treated as a muscle mass in the embodiment. In the embodiment, an average value of thicknesses of the muscle layer calculated from the time-series waveform data received by the eight reception elements 812 to 882 may be used as a thickness of a muscle layer of a specific target site. In another embodiment, a thickness of the muscle layer calculated from the time-series waveform data received from a specific one of the eight reception elements 812 to 882 may be used as the thickness of the muscle layer of the specific target site.

Next, the load specifying device 100 will be described with reference to FIG. 1. The load specifying device 100 includes a processor 110, a storage device 120, an interface circuit 130, and an input device 140 and a display unit 150 coupled to the interface circuit 130. The load specifying device 100 is, for example, a personal computer. The load specifying device 100 is a device that uses a trained machine learning model 122 stored in the storage device 120 to specify an appropriate load to a user who performs muscle strength training using the load device 300.

The processor 110 includes a training execution unit 112, a spectrum processing unit 113, a waveform acquisition unit 116, and a specifying unit 118 by executing various programs stored in the storage device 120. The training execution unit 112 executes training processing of the machine learning model 122 using a reference data group TDG, which is also a collection of training sets SM.

The waveform acquisition unit 116 acquires the time-series waveform data related to a fascia that is generated by the sensor device 400, and stores the time-series waveform data in the storage device 120. When the time-series waveform data is time-series waveform data for each of a plurality of subjects, the time-series waveform data is stored in the storage device 120 as an element of the reference data group TDG. When the time-series waveform data is time-series waveform data of a user, the time-series waveform data is stored in the storage device 120 as an evaluation data group EDG. The user determines an optimal load based on the time-series waveform data for each of the plurality of subjects and performs the muscle strength training.

Each piece of time-series waveform data in the reference data group TDG is data related to a fascia that is generated for each subject by the sensor device 400 when the subject performs the muscle strength training by setting a reference load on the load device 300 such that a muscle gain amount of the subject per unit time satisfies a predetermined condition. Each piece of time-series waveform data in the reference data group TDG can be measured during a period when the subject is performing the muscle strength training. The predetermined condition is a condition that the muscle gain amount in the measurement target site per hour is equal to or greater than a reference gain amount. In the embodiment, the predetermined condition is a condition that the muscle gain amount per unit time is maximum. The muscle gain amount in the specific target site is, for example, a difference in muscle mass that is calculated using the sensor device 400 before and after performing the muscle strength training for a predetermined time with a plurality of loads in stages for each subject. Each piece of time-series waveform data in the reference data group TDG is associated with load data LD indicating a load value of the load device 300, which is a source for generating the time-series waveform data.

Each piece of time-series waveform data in the evaluation data group EDG is time-series waveform data for each of different candidate loads when a user executes a plurality of types of muscle strength training for which the different candidate loads are set on the load device 300. The time-series waveform data constituting the evaluation data group EDG is also referred to as target time-series waveform data. As described above, the user performs the muscle strength training by changing the candidate load in stages from within a predetermined load range. The target time-series waveform data generated by the sensor device 400 for each candidate load is acquired by the waveform acquisition unit 116 and stored in the storage device 120 as the evaluation data group EDG. Each piece of target time-series waveform data in the evaluation data group EDG is associated with a generation condition under which the target time-series waveform data is generated. In the embodiment, the generation condition includes at least the load data LD for specifying a magnitude of the load generated by the load generation unit 320. The generation condition may include either a site identifier for identifying a measurement target site of a person to be measured by the ultrasonic probe 410 or a load device identifier for identifying the load device 300.

Each piece of time-series waveform data in the evaluation data group EDG and each piece of time-series waveform data in the reference data group TDG are generated from the same specific target site of the person.

The spectrum processing unit 113 acquires a feature spectrum Sp from output of a specific layer of the machine learning model 122 by inputting the time-series waveform data to the trained machine learning model 122. Details of the feature spectrum Sp will be described later. The spectrum processing unit 113 includes a first spectrum acquisition unit 114 and a second spectrum acquisition unit 115.

The first spectrum acquisition unit 114 inputs the time-series waveform data in the reference data group TDG, that is, a plurality of pieces of time-series waveform data related to fascias that correspond to the plurality of subjects, to the trained machine learning model 122 one by one. The first spectrum acquisition unit 114 acquires a known feature spectrum KSp as the feature spectrum Sp from the output of the specific layer of the trained machine learning model 122 for each of the plurality of pieces of time-series waveform data included in the reference data group TDG. A plurality of acquired known feature spectra KSp are stored in the storage device 120 as elements of a known feature spectrum group KSpG.

The second spectrum acquisition unit 115 sequentially inputs a plurality of pieces of target time-series waveform data included in the evaluation data group EDG to the trained machine learning model 122 one by one. The second spectrum acquisition unit 115 acquires a target feature spectrum ESp as the feature spectrum Sp from the output of the specific layer of the trained machine learning model 122 for each of the plurality of pieces of target time-series waveform data included in the evaluation data group EDG. A plurality of acquired target feature spectra ESp are stored in the storage device 120 as elements of a target feature spectrum group ESpG.

The specifying unit 118 includes a calculation unit 117 and a specification processing unit 119. The calculation unit 117 calculates a spectrum similarity RSp, which is a similarity between each of the plurality of pieces of known feature spectra KSp included in the known feature spectrum group KSpG and each of the plurality of pieces of target feature spectra ESp included in the target feature spectrum group ESpG. A specific method of calculating the spectrum similarity RSp will be described later. The specification processing unit 119 specifies the spectrum similarity RS S satisfying a predetermined extraction condition among a plurality of spectrum similarities RSp calculated by the calculation unit 117. The specification processing unit 119 specifies a candidate load corresponding to the target time-series waveform data as a calculation source of the specified spectrum similarity RSp. In the embodiment, the predetermined extraction condition is a condition that the spectrum similarity RSp is the highest among the plurality of spectrum similarities RSp. The specification processing unit 119 may transmit, to the load device 300, specific load information indicating the specified candidate load. The load control unit 310 may set a load of the load generation unit 320 to be a load value indicated by the specific load information. In another embodiment, the predetermined extraction condition may include a condition that the spectrum similarity RSp is equal to or greater than a threshold value.

In the above, at least part of functions of each functional unit of the processor 110, such as the training execution unit 112 and the spectrum processing unit 113, may be implemented by a hardware circuit. The processor 110 in the present description is a term including such a hardware circuit.

The input device 140 is a device for a person such as a subject or a user to input information to the load specifying device 100. The input device 140 is, for example, a keyboard, a mouse, or a touch panel. The display unit 150 displays various types of information. The display unit 150 is, for example, a liquid crystal monitor. The interface circuit 130 is an interface for exchanging information between the display unit 150 and the input device 140 and the processor 110 and the storage device 120.

The storage device 120 stores the trained machine learning model 122, the reference data group TDG, the evaluation data group EDG, the known feature spectrum group KSpG, and the target feature spectrum group ESpG.

The trained machine learning model 122 is a vector neural network-based machine learning model having a plurality of vector neuron layers. The trained machine learning model 122 is trained by, for example, adjusting parameters and the like using the reference data group TDG. A configuration example and operation of the trained machine learning model 122 will be described later. In another embodiment, the trained machine learning model 122 may be trained using training data generally used for training a machine learning model.

The reference data group TDG includes the time-series waveform data generated by the muscle strength training of the subject, and the load data LD indicating the reference load during the muscle strength training and associated with the time-series waveform data. The reference data group TDG does not need to include the load data LD.

The evaluation data group EDG includes the target time-series waveform data generated by the muscle strength training of the user and the load data LD indicating the candidate load during the muscle strength training and associated with the target time-series waveform data.

Since the known feature spectrum group KSpG and the target feature spectrum group ESpG have already been described, a description thereof will be omitted here.

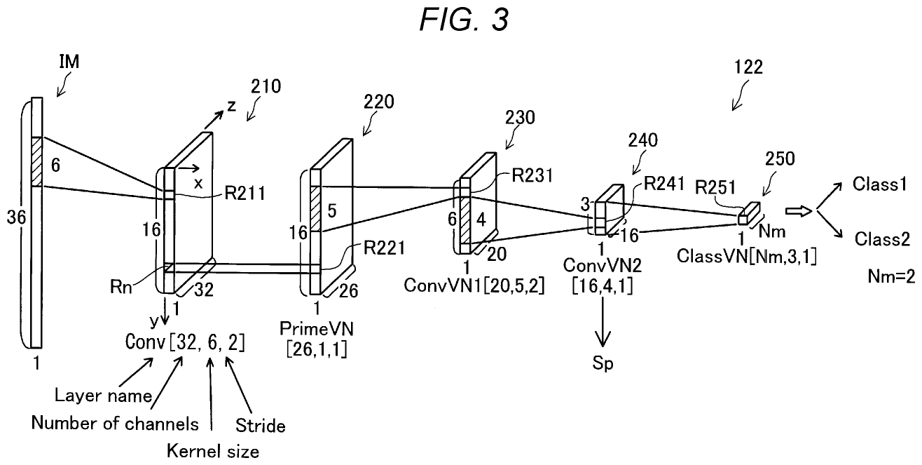

FIG. 3 is a diagram showing a configuration of the machine learning model 122. The machine learning model 122 includes, in order from an input data IM side, a convolutional layer 210, a prime vector neuron layer 220, a first convolutional vector neuron layer 230, a second convolutional vector neuron layer 240, and a classification vector neuron layer 250 as an output layer. Among the five layers 210 to 250, the convolutional layer 210 is a lowermost layer, and the classification vector neuron layer 250 is an uppermost layer. In the following description, the layers 210 to 250 are also referred to as “Conv layer 210”, “PrimeVN layer 220”, “ConvVN1 layer 230”, “ConvVN2 layer 240”, and “classVN layer 250”, respectively.

In the embodiment, since the input data IM is the time-series waveform data, the input data IM is data in a one-dimensional array. For example, the input data IM is data indicating an intensity of the reflected waves received by the reception elements 812 to 882 for each time.

In the example in FIG. 3, two convolutional vector neuron layers 230 and 240 are used, but the number of convolutional vector neuron layers is any number, and the convolutional vector neuron layer may be omitted. It is preferable to use one or more convolutional vector neuron layers.

Configurations of the layers 210 to 250 in FIG. 3 can be described as follows.

### Description of Configuration of Machine Learning Model 122

Conv layer 210: Conv [32, 6, 2]
PrimeVN layer 220: PrimeVN [26, 1, 1]
ConvVN1 layer 230: ConvVN1 [20, 5, 2]
ConvVN2 layer 240: ConvVN2 [16, 4, 1]
classVN layer 250: classVN [Nm, 3, 1]
vector dimension VD: VD=16

In the description of the layers 210 to 250, a character string before parentheses is a layer name, and numbers in the parentheses are the number of channels, a kernel size, and a stride in this order. For example, the layer name of the Conv layer 210 is “Conv”, the number of channels is 32, the kernel size is 1×6, and the stride is 2. In FIG. 3, this description is shown below each layer. A hatched rectangle drawn in each layer represents a kernel size used to calculate an output vector of an adjacent upper layer. In the embodiment, since the data IM is data in a one-dimensional array, the kernel size is also one-dimensional. Values of parameters used in the description of the layers 210 to 250 are merely examples, and can be freely changed.

The Conv layer 210 is a layer composed of scalar neurons. The other four layers 220 to 250 are layers composed of vector neurons. The vector neuron is a neuron whose input and output are vectors. In the above description, the number of dimensions of an output vector of each vector neuron is constant at 16. Hereinafter, a term “node” is used as a broader concept of the scalar neuron and the vector neuron.

In FIG. 3, for the Conv layer 210, a first axis x and a second axis y that define plane coordinates of a node array and a third axis z indicating a depth are shown. Sizes of the Conv layer 210 in x, y, and z directions being 1, 16, and 32 are also shown. The size in the x direction and the size in the y direction are referred to as “resolution”. In the embodiment, the resolution in the x direction is always 1. The size in the z direction is the number of channels. The three axes x, y, and z are used as coordinate axes indicating positions of nodes even in other layers. In FIG. 3, in the layers other than the Conv layer 210, illustration of these axes x, y, and z is omitted.

Resolution W1 after convolution in the y direction is given by the following equation:

$$W 1 = Ceil \{ ( W 0 - Wk + 1 ) / S \} \qquad ( 1 )$$

in which W0 is resolution before convolution, Wk is the kernel size, S is the stride, and Ceil{X} is a function for performing an operation of rounding up decimals of X.

The resolution of each layer shown in FIG. 3 is an example when the resolution of the data IM in the y direction is 36, and actual resolution of each layer is appropriately changed according to a size of the data IM.

The classVN layer 250 has Nm channels. In the example in FIG. 3, Nm=2. Generally, Nm is an integer equal to or greater than 2, and is the number of known classes that can be discriminated using the machine learning model 122. The number Nm of discriminable classes can be set to a different value for each machine learning model 122. Determination values class 1 and class 2 for the two known classes are output from two channels of the classVN layer 250. Normally, a class having the largest value of the determination values class 1 and class 2 is used as a class discrimination result of the data IM.

In FIG. 3, partial regions Rn in the layers 210, 220, 230, 240, and 250 are further drawn. An additional letter “n” of the partial region Rn is a sign of each layer. For example, a partial region R210 indicates a partial region in the Conv layer 210. The “partial region Rn” is a region that is specified by a plane position (x, y) defined by a position in the first axis x and a position in the second axis y in each layer and includes a plurality of channels along the third axis z. The partial region Rn has dimensions of “width”×“height”×“depth” corresponding to the first axis x, the second axis y, and the third axis z. In the embodiment, the number of nodes in one “partial region Rn” is “1×1×the number of depths”, that is, “1×1×the number of channels”.

As shown in FIG. 3, the feature spectrum Sp is calculated and acquired by the spectrum processing unit 113 from output of the ConvVN2 layer 240 as a specific layer of the trained machine learning model 122. When the input data IM is the time-series waveform data in the reference data group TDG, the acquired feature spectrum Sp is stored in the known feature spectrum group KSpG. When the input data IM is the time-series waveform data in the evaluation data group EDG, the acquired feature spectrum Sp is stored in the target feature spectrum group ESpG. The specific layer of the trained machine learning model 122 may be any one of the ConvVN1 layer 230, the ConvVN2 layer 240, and the classVN layer 250.

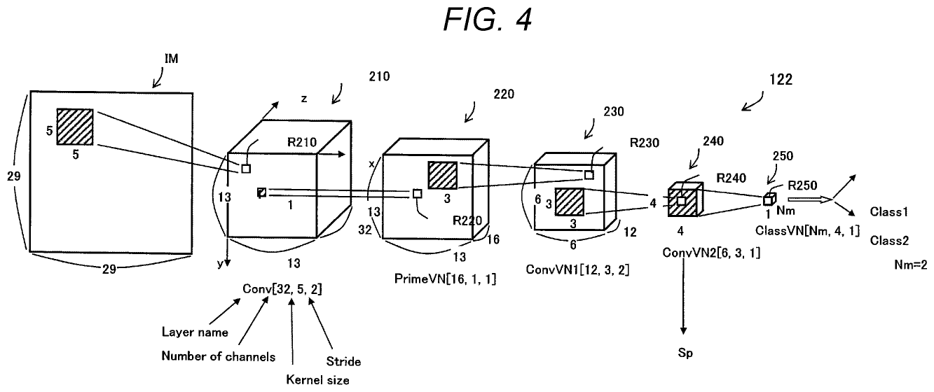

FIG. 4 is a diagram showing another configuration of the trained machine learning model 122. The trained machine learning model 122 is different from the trained machine learning model 122 using the data IM in a one-dimensional array in FIG. 3 in that the input data IM is data in a two-dimensional array such as image data. Configurations of the layers 210 to 250 in FIG. 4 can be described as follows.

### Description of Configuration of Each Layer

Conv layer 210: Conv [32, 5, 2]
PrimeVN layer 220: PrimeVN [16, 1, 1]
ConvVN1 layer 230: ConvVN1 [12, 3, 2]
ConvVN2 layer 240: ConvVN2 [6, 3, 1]
classVN layer 250: classVN [Nm, 4, 1]
vector dimension VD: VD=16

The trained machine learning model 122 shown in FIG. 4 can be used, for example, in a discrimination system that performs class discrimination of a discrimination target image. However, in the following description, the trained machine learning model 122 shown in FIG. 3 is used.

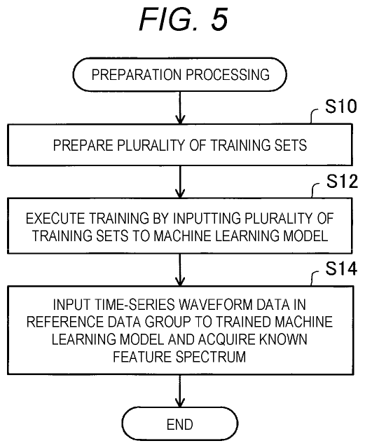

FIG. 5 is a diagram showing processing of generating the trained machine learning model 122. A process of generating the trained machine learning model 122 by training an untrained machine learning model will be described with reference to FIG. 5. First, in step S10, a plurality of training sets SM are prepared.

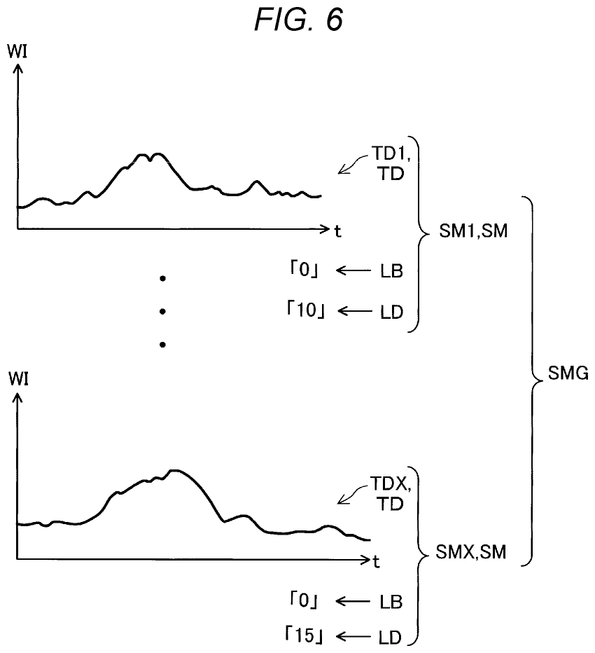

FIG. 6 is a diagram showing the plurality of training sets SM. The training set SM according to the embodiment includes training data TD, a pre-label LB associated with the training data TD, and the load data LD associated with the training data TD. When a plurality of training sets SM1 to SMX are used without being distinguished, they are referred to as the training set SM. The plurality of training sets SM1 to SMX are also collectively referred to as a training set group SMG. In the embodiment, each piece of time-series waveform data in the reference data group TDG is used for each piece of training data TD. As described above, the time-series waveform data is data in which reflection intensities WI are arranged for each time t during a predetermined time during which muscle strength training is performed. In the pre-label LB, a label “0” indicating “optimal” as the muscle strength training is associated with each piece of training data TD. As described in the reference data group TDG, the load data LD is data indicating a load value of the load device 300, which is the source for generating the training data TD. The training set group SMG may be stored in the storage device 120, and in this case, a collection of each piece of time-series waveform data in the training set group SMG and the load data LD constitutes the reference data group TDG. As described above, step S10 is a step of preparing the time-series waveform data related to a fascia of a subject for each of a plurality of subjects when the subject performs the muscle strength training for which the reference load is set such that the muscle gain amount of the subject per unit time satisfies the predetermined condition.

As shown in FIG. 5, in step S12 after step S10, the training execution unit 112 executes training by inputting the plurality of training sets SM1 to SMX constituting the training set group SMG to the machine learning model before training. Specifically, the training execution unit 112 executes training of the machine learning model so as to reproduce a correspondence between the training data TD as the data IM and the pre-label LB associated with the training data TD. When the training of the machine learning model is completed, the trained machine learning model 122 is stored in the storage device 120.

Next, in step S14, the first spectrum acquisition unit 114 inputs the time-series waveform data constituting the reference data group TDG to the trained machine learning model 122, and acquires the known feature spectrum KSp from the output of the ConvVN2 layer 240 as the specific layer for each of the plurality of pieces of time-series waveform data.

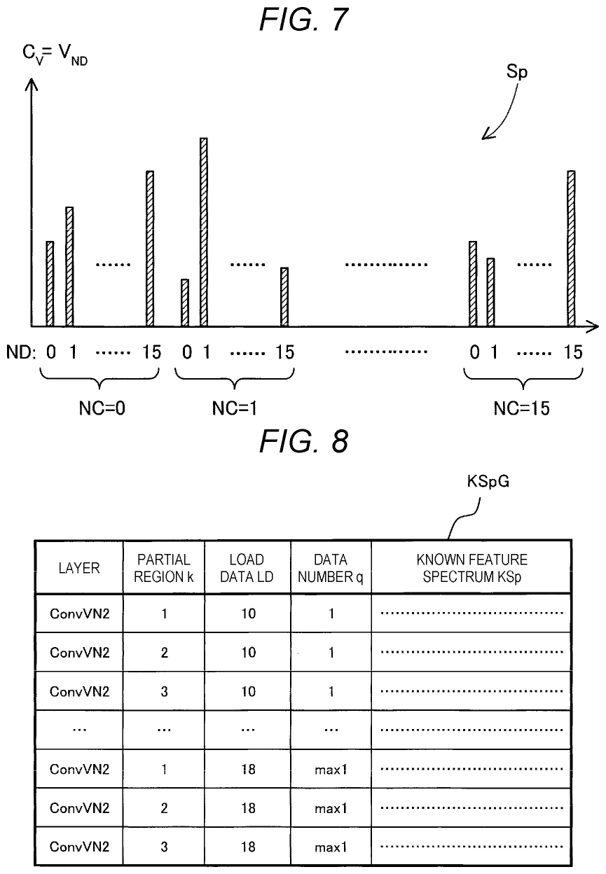

FIG. 7 is a diagram showing the feature spectrum Sp obtained by inputting the time-series waveform data as the data IM to the trained machine learning model 122. As shown in FIG. 3, in the embodiment, the feature spectrum Sp is acquired by being generated from the output of the ConvVN2 layer 240. A horizontal axis in FIG. 7 is a position of a vector element related to output vectors of a plurality of nodes included in one partial region R240 of the ConvVN2 layer 240. The position of the vector element is represented by a combination of an element number ND of the output vector in each node and a channel number NC. In the embodiment, since the number of vector dimensions is 16, that is, the number of elements of the output vector output by each node is 16, the element numbers ND of the output vector are 16 from 0 to 15. Since the number of channels of the ConvVN2 layer 240 is 16, the channel number NC is 16 from 0 to 15. In other words, the feature spectrum Sp is obtained by arranging a plurality of element values of the output vector of each vector neuron included in one partial region R240 over a plurality of channels along the third axis z.

A vertical axis in FIG. 7 indicates a feature value C_{V} at each spectrum position. In this example, the feature value C_{V} is a value V_{ND} of each element of the output vector. The feature value C_{V} may be subjected to statistical processing such as centering to an average value 0. As the feature value C_{V}, a value obtained by multiplying the value V_{ND} of each element of the output vector by a normalization coefficient may be used, or the normalization coefficient may be used as it is. In a latter case, the number of feature values C_{V} included in the feature spectrum Sp is equal to the number of channels and is 16. The normalization coefficient is a value corresponding to a vector length of an output vector of a node.

The number of feature spectra Sp obtained from output of the ConvVN2 layer 250 for one piece of data IM is 3, which is equal to the number of plane positions (x, y) of the ConvVN2 layer 240, that is, the number of partial regions R240.

FIG. 8 is a diagram showing a configuration of the known feature spectrum group KSpG. In this example, the known feature spectrum group KSpG having the known feature spectrum KSp obtained from the output of the ConvVN2 layer 240 as a constituent element is shown.

Individual records of the known feature spectrum group KSpG include a parameter k indicating an order of the partial regions Rn in a layer, the load data LD, a parameter q indicating a data number for identifying the input data IM, and the known feature spectrum KSp. The known feature spectrum KSp is the same as the feature spectrum Sp in FIG. 7.

The parameter k of the partial region Rn takes a value indicating which one a plurality of partial regions Rn, that is, which of a plane position (x, y) included in a specific layer is selected. In the ConvVN2 layer 240, since the number of partial regions R240 is 3, k=1 to 3. The load data LD indicates a magnitude of the load set by the load control unit 310 when the time-series waveform data as a generation source of the known feature spectrum KSp is acquired. The parameter q of the data number is a number for identifying the time-series waveform data as the input data IM for acquiring the known feature spectrum KSp, and the number of pieces of the time-series waveform data in the reference data group TDG takes a maximum number max1. In the embodiment, the parameter q takes a value from 1 to max1. The known feature spectrum group KSpG may further include, for each known feature spectrum KSp, a class parameter for identifying a class, which is the pre-label LB.

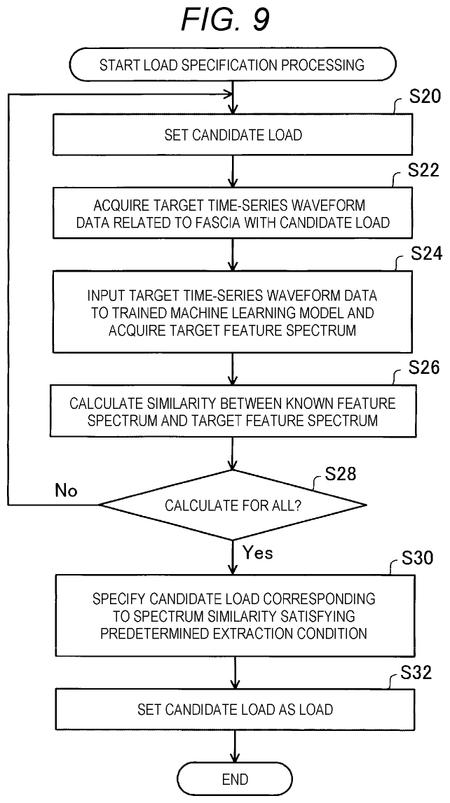

FIG. 9 is a flowchart of load specifying processing executed by the load specifying system 5. In the load specifying processing, when the user performs the muscle strength training using the load device 300, an optimal load at which a muscle gain amount is maximum is specified. Before executing the load specifying processing, candidate load conditions for acquiring the target feature spectrum ESp during the muscle strength training are set in the load specifying device 100. In the embodiment, as the candidate load conditions, a lower limit value and an upper limit value of the candidate load, and an increase value when the load is increased in stages from the lower limit value to the upper limit value at a certain interval are determined. In another embodiment, as the candidate load conditions, a lower limit value and an upper limit value of the candidate load, and a decrease value when the load is reduced in stages from the upper limit value to the lower limit value at a certain interval are determined. In still another embodiment, the candidate load conditions may be a collection of a plurality of candidate loads.

In step S20, the load control unit 310 sets one candidate load. Specifically, by receiving an instruction to set a candidate load from the outside via the input unit 330 shown in FIG. 1, the load control unit 310 sets the candidate load as a load.

Next, in step S22 shown in FIG. 9, the waveform acquisition unit 116 acquires, from the sensor device 400, the target time-series waveform data related to the fascia of the measurement target site when the user uses the load device 300 to execute the muscle strength training for a predetermined time with the candidate load set in step S20. The predetermined time which is a user muscle strength training time is the same as a predetermined time which is a time for the muscle strength training of the subject performed to acquire the time-series waveform data of the subject. The measurement target site is the same between the subject and the user. The target time-series waveform data acquired in step S22 is stored in the storage device 120 as an element of the evaluation data group EDG.

Next, in step S24, the second spectrum acquisition unit 115 inputs the target time-series waveform data to the trained machine learning model 122, and acquires the target feature spectrum ESp from the output of the ConvVN2 layer 240 as the specific layer. The acquired target feature spectrum ESp is associated with the load data LD and stored in the target feature spectrum group ESpG. A data configuration of the target feature spectrum group ESpG is the same as a data configuration of the known feature spectrum group KSpG shown in FIG. 8, except that the known feature spectrum KSp is replaced with the target feature spectrum ESp.

Next, in step S26, the calculation unit 117 calculates the spectrum similarity RSp between each of the plurality of known feature spectra KSp constituting the known feature spectrum group KSpG and the target feature spectrum ESp acquired in step S24. The calculated spectrum similarity RSp is stored in the storage device 120 together with the load data LD associated with the target feature spectrum ESp.

Next, in step S28, the specifying unit 118 determines whether the spectrum similarity RSp is calculated for all the target feature spectra ESp corresponding to all the candidate loads represented by the candidate load conditions.

In step S28, when the spectrum similarity RSp is not calculated for all the target feature spectra ESp corresponding to all the candidate loads, the load control unit 310 sets one of the remaining candidate loads in step S20. In the embodiment, until the spectrum similarity RSp is calculated for all the target feature spectra ESp, the load control unit 310 increases the magnitude of the load in stages at a certain interval based on the candidate load conditions. That is, the different candidate loads set in step S20 differ in magnitude of the loads at a certain interval. The processing from step S20 to step S26 is repeatedly executed until the spectrum similarity RSp is calculated for all the target feature spectra ESp.

In step S28, when the spectrum similarity RSp is calculated for all the target feature spectra ESp, step S30 is executed by the specification processing unit 119. In step S30, the specification processing unit 119 specifies the spectrum similarity RSp satisfying the predetermined extraction condition among the plurality of spectrum similarities RSp calculated by repeatedly executing step S20 to step S26, and specifies the candidate load corresponding to the target time-series waveform data as the calculation source of the specified spectrum similarity RSp. Specifically, the specification processing unit 119 specifies the candidate load indicated by the load data LD by specifying the load data LD associated with the target time-series waveform data as the calculation source with reference to the target feature spectrum group ESpG shown in FIG. 1. In the embodiment, the predetermined extraction condition is a condition that the spectrum similarity RSp is the highest among the plurality of spectrum similarities RSp. Accordingly, the specification processing unit 119 specifies one candidate load corresponding to the highest spectrum similarity RSp. The load data LD indicating the specified candidate load may be transmitted to the load device 300 wired or wirelessly, or may be displayed on the display unit 150.

Next, in step S32, the load control unit 310 sets the specified candidate load as the load of the load generation unit 320. For example, when receiving the load data LD indicating the candidate load specified in step S30 from the load specifying device 100 wired or wirelessly, the load control unit 310 may execute step S32 by setting the load of the load generation unit 320 to a load value indicated by the load data LD. For example, step S32 may be executed when a user or a trainer refers to the load data LD displayed on the display unit 150 and inputs the load value indicated by the load data LD to the load device 300, and the load control unit 310 sets the load corresponding to the input load value to the load generation unit 320. In another embodiment, the load specifying processing may omit step S32.

For example, in a case where the predetermined extraction condition in step S28 is a condition that the spectrum similarity RSp is equal to or greater than a threshold value, or the like, when there are a plurality of candidate loads specified in step S30, the specification processing unit 119 may display the plurality of specified candidate loads on the display unit 150. In this case, for example, the specification processing unit 119 may display candidate load information indicating values of the plurality of candidate loads and similarity information indicating the spectrum similarity RSp associated with each piece of candidate load information on the display unit 150. In this way, the user can easily select a load appropriate for the muscle strength training of the user by referring to the spectrum similarity RSp.

As described above, in the load specifying processing, for example, the following processing 1 and processing 2 are executed by repeatedly executing a processing routine of step S20 to step S26 until the spectrum similarity RSp is calculated for all the target feature spectra ESp.

Step 1: acquiring the target time-series waveform data for each of the different candidate loads when the user executes a plurality of types of muscle strength training for which the different candidate loads are set, which is a step of repeatedly executing step S22.

Step 2: inputting the plurality of pieces of target time-series waveform data to the trained machine learning model 122 and acquiring the plurality of target feature spectra ESp from the output of the ConvVN2 layer 240 as the specific layer, which is a step of repeatedly executing step S24.

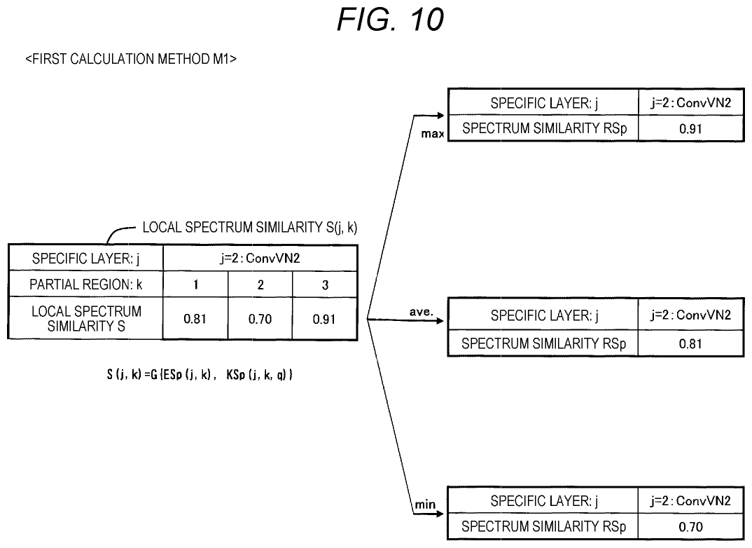

Next, a method of calculating the spectrum similarity RSp will be described with reference to FIGS. 10 to 12. FIG. 10 is a diagram showing a first calculation method M1 of the spectrum similarity RSp. In the first calculation method M1, first, a local spectrum similarity S(j, k) is calculated for each partial region Rn from the output of the ConvVN2 layer 240 as the specific layer.

In the first calculation method M1, the local spectrum similarity S(j, k) is calculated using the following equation:

$$S ( j , k ) = G \{ ESp ( j , k ) , KSp ( j , k , q ) \} \qquad ( c 1 )$$

in which
j is a parameter indicating the specific layer,
k is a parameter indicating the partial region Rn,
q is a parameter indicating the data number,
G{a, b} is a function for obtaining a spectrum similarity between a and b,
ESp (j, k) is a target feature spectrum obtained from the output of the specific partial region Rn of the specific layer j, and
KSp (j, k, q) is a known feature spectrum of the data number q obtained from the output of the specific partial region Rn of the specific layer j in the known feature spectrum group KSpG.

As the function G{a, b} for obtaining the local spectrum similarity, for example, an equation for obtaining a cosine similarity or an equation for obtaining a similarity according to a distance can be used.

Three types of spectrum similarity RSp shown on the right side in FIG. 10 are calculated as representative similarities by statistically processing the local spectrum similarity S (j, k) for the plurality of partial regions Rn. The statistical processing is performed by taking a maximum value, an average value, or a minimum value of the plurality of local spectrum similarities S(j, k). Although not shown, which of the maximum value, the average value, and the minimum value is used for calculation is experimentally or empirically set in advance by the user.

As described above, in the first calculation method M1 of the spectrum similarity RSp, the spectrum similarity RSp is calculated by the following method.
(1) The local spectrum similarity S (j, k), which is a spectrum similarity between the target feature spectrum ESp obtained from the output of the specific partial region Rn of the specific layer j and the known feature spectrum KSp obtained from the output of the specific partial region Rn of the specific layer j, is obtained in one piece of target time-series waveform data, and
(2) the spectrum similarity RSp is obtained by taking the maximum value, the average value, or the minimum value of the local spectrum similarity S(j, k) for the plurality of partial regions Rn.

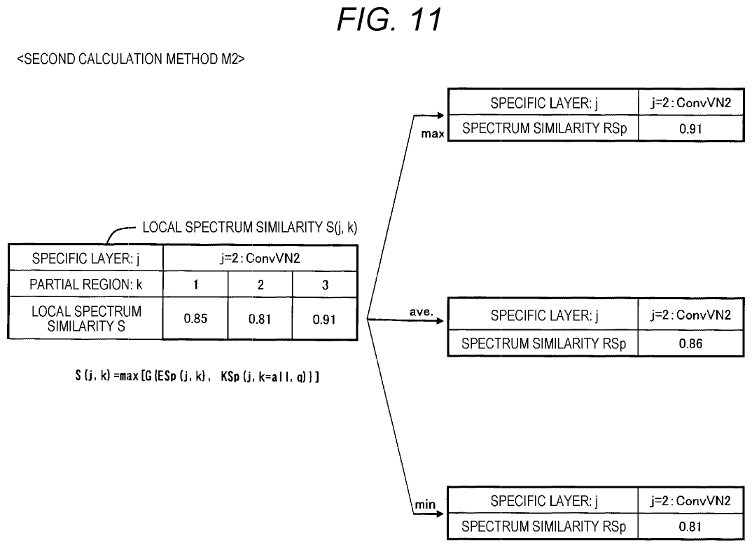

FIG. 11 is a diagram showing a second calculation method M2 of the spectrum similarity RSp. The calculation unit 117 calculates a local spectrum similarity S (i, j, k) using the following equation instead of the above equation (c1):

$$S ( j , k ) = max [ G \{ ESp ( j , k ) , KSp ( j , k = all , q ) \} ] \qquad ( c 2 )$$

in which
KSp (j, k=all, q) is the known feature spectrum KSp of the data number q that is obtained from the output for each partial region Rn of the specific layer j in the known feature spectrum group KSpG. Max [G(a, b)] indicates a spectrum similarity having a maximum value among the calculated spectrum similarities.

In the first calculation method M1 described above, a comparison target between the target feature spectrum ESp and the known feature spectrum KSp is the same partial region Rn, but in a second calculation method M2, the target feature spectrum ESp in one partial region Rn and the known feature spectra KSp in all partial regions Rn are to be calculated. Other methods in the second calculation method M2 are the same as in the first calculation method M1.

As described above, in the second calculation method M2 of the spectrum similarity, the spectrum similarity RSp is calculated by the following method.
(1) A plurality of spectrum similarities between the target feature spectrum ESp obtained from the output of the specific partial region Rn of the specific layer j and the known feature spectrum KSp of all the partial regions Rn of the specific layer j are obtained in one piece of target time-series waveform data, and a spectrum similarity having a maximum value among the plurality of spectrum similarities is obtained as the local spectrum similarity S (j, k) of the partial region Rn, and
(2) the spectrum similarity RSp is obtained by taking the maximum value, the average value, or the minimum value of the local spectrum similarity S(j, k) for the plurality of partial regions Rn.

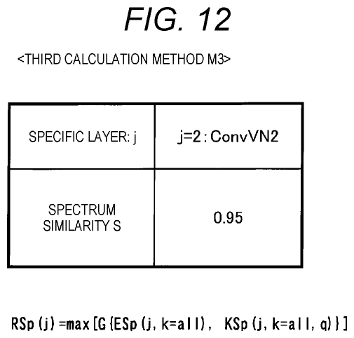

FIG. 12 is a diagram showing a third calculation method M3 of the spectrum similarity. In the third calculation method M3, the spectrum similarity RSp is calculated from the output of the ConvVN2 layer 240 as the specific layer, without obtaining the local spectrum similarity S (j, k).

A spectrum similarity RSp (j) obtained by the third calculation method M3 is calculated using the following equation:

$$RSp ( j ) = max [ G \{ ESp ( j , k = all ) , KSp ( j , k = all , q ) \} ] \qquad ( c3 )$$

In which
Sp (j, k=all) is a feature spectrum obtained from output of all the partial regions Rn of the specific layer j.

As described above, in the third calculation method M3 of the spectrum similarity RSp, the spectrum similarity RSp is calculated by the following method.
(1) Individual spectrum similarities, which are similarities between the target feature spectrum ESp corresponding to all partial regions obtained from the output of the specific layer j and the known feature spectra KSp corresponding to all partial regions Rn of all data numbers associated with the specific layer j are obtained in one piece of target time-series waveform data, and
(2) a spectrum similarity having a maximum value among the plurality of individual spectrum similarities is defined as the spectrum similarity RSp.

According to the above-described embodiment, as shown in FIG. 9, the spectrum similarity RSp satisfying the extraction condition is specified among the spectral similarities RSp between each of the plurality of known feature spectra KSp and each of the plurality of target feature spectra ESp for each of the different candidate loads. The load specifying device 100 specifies the candidate load corresponding to the target time-series waveform data as the calculation source of the specified spectrum similarity RSp. Accordingly, the appropriate load can be specified for various users, and thus the specified appropriate load can be set as the load for the muscle strength training.

According to the above-described embodiment, the known feature spectrum KSp to be compared with the target feature spectrum ESp can be acquired based on the time-series waveform data when the subject performs the muscle strength training for which the reference load is set on the load device 300 such that the muscle gain amount of the subject per unit time satisfies the predetermined condition. Accordingly, the load specifying device 100 can accurately specify the candidate load at which the muscle gain amount of the user per unit time is equal to or greater than the reference gain amount, based on the spectrum similarity RSp. Accordingly, by setting the candidate load on the load device 300 as the load for the muscle strength training of the user, the muscle strength training in which the muscle gain amount per unit time is equal to or greater than the reference gain amount can be executed more reliably. Particularly, in the embodiment, the predetermined condition is a condition that the muscle gain amount per unit time is the maximum, and thus the candidate load at which the muscle gain amount of the user per unit time is the maximum can be accurately specified. Accordingly, by setting the candidate load as the load for the muscle strength training of the user, the muscle strength training in which the muscle gain amount per unit time is the maximum can be executed more reliably. According to the above-described embodiment, the load control unit 310 can easily set the appropriate load for the muscle strength training for the user by setting the specified candidate load as the load of the load generation unit 320 according to step S32 in FIG. 9. According to the above-described embodiment, the different candidate loads set in step S20 in FIG. 9, which is repeatedly executed, differ in magnitude of the loads at a certain interval. Accordingly, the candidate load can be changed in stages, and thus an appropriate load can be more accurately specified for the user.

### B. Other Embodiments

In the above-described embodiment, the time-series waveform data may be measured at one measurement target site or at a plurality of measurement target sites. When the time-series waveform data is acquired at the plurality of measurement target sites, site information indicating the measurement target site may be associated with the time-series waveform data or the feature spectrum Sp acquired from the time-series waveform data, and may be stored in the storage device 120. In this case, the spectrum similarity RSp is calculated by comparing the known feature spectrum KSp with the target feature spectrum ESp, which have the same site information. In this way, an appropriate load for muscle strength training can be easily specified for each muscle part of the user, such as abdominal muscles and biceps brachii muscles.

### C. Other Embodiments

The present disclosure is not limited to the above embodiments, and can be implemented in various forms without departing from the spirit of the present disclosure. For example, the present disclosure can be implemented by the following aspects. In order to solve a part of or all of problems of the present disclosure, or to achieve a part of or all of effects of the present disclosure, technical features of the above embodiments corresponding to technical features in each of the following aspects can be replaced or combined as appropriate. Technical characteristics can be deleted as appropriate unless described as essential in the present specification.

(1) According to a first aspect of the present disclosure, a load specifying method for a muscle strength training device is provided. The load specifying method includes: a step (a) of preparing, for each of a plurality of subjects, time-series waveform data related to a fascia of the subject when the subject performs muscle strength training for which a reference load is set such that a muscle gain amount of the subject per unit time satisfies a predetermined condition; a step (b) of inputting a plurality of pieces of the time-series waveform data to a vector neural network-based trained machine learning model having a plurality of vector neuron layers and acquiring a known feature spectrum as a feature spectrum from output of a specific layer of the trained machine learning model for each of the plurality of pieces of time-series waveform data; a step (c) of acquiring target time-series waveform data, which is the time-series waveform data for each of different candidate loads, when a user executes a plurality of types of muscle strength training for which the different candidate loads are set; a step (d) of inputting a plurality of pieces of the target time-series waveform data to the trained machine learning model and acquiring a target feature spectrum as the feature spectrum from the output of the specific layer for each of the plurality of pieces of target time-series waveform data; and a step (e) of calculating a spectrum similarity, which is a similarity between each of a plurality of the known feature spectra and each of a plurality of the target feature spectra, specifying the spectrum similarity satisfying a predetermined extraction condition among a plurality of the calculated spectrum similarities, and specifying the candidate load corresponding to the target time-series waveform data as a calculation source of the specified spectrum similarity. According to this aspect, the spectrum similarity satisfying the extraction condition is specified among the spectrum similarity between each of the plurality of known feature spectra and each of the plurality of target feature spectra for the different candidate loads, and the candidate load corresponding to the target time-series waveform data as a calculation source of the specified spectrum similarity is specified. Accordingly, the appropriate load can be specified for various users, and thus the specified appropriate load can be set as the load for the muscle strength training.

(2) In the above aspect, in the step (a), the predetermined condition may be a condition that the muscle gain amount per unit time is equal to or greater than a reference gain amount, and in the step (e), the extraction condition may be a condition that the spectrum similarity is equal to or greater than a threshold value. According to this aspect, the candidate load at which the muscle gain amount of the user per unit time is equal to or greater than the reference gain amount can be accurately specified. Accordingly, by setting the candidate load as the load for the muscle strength training of the user, the muscle strength training in which the muscle gain amount per unit time is equal to or greater than the reference gain amount can be executed more reliably.

(3) In the above aspect, in the step (a), the predetermined condition may be a condition that the muscle gain amount per unit time is maximum, and in the step (e), the extraction condition may be a condition that the spectrum similarity is highest among the plurality of spectrum similarities. According to this aspect, the candidate load at which the muscle gain amount of the user per unit time is maximum can be accurately specified. Accordingly, by setting the candidate load as the load for the muscle strength training of the user, the muscle strength training in which the muscle gain amount per unit time is the maximum can be executed more reliably.

(4) In the above aspect, the step (e) includes setting the specified candidate load as a load when the user performs the muscle strength training. According to this aspect, an appropriate muscle strength training load for the user can be easily set.

(5) In the above aspect, in the step (c), the different candidate loads may differ in magnitude of the loads at a certain interval. According to this aspect, since the different candidate loads differ in magnitude of the loads at a certain interval, an appropriate load for the user can be more accurately specified.

(6) According to a second aspect of the present disclosure, a load specifying device for a muscle strength training device is provided. The load specifying device includes: a storage device configured to store a vector neural network-based trained machine learning model having a plurality of vector neuron layers; a first spectrum acquisition unit configured to, when a subject performs muscle strength training for which a reference load is set such that a muscle gain amount of the subject per unit time satisfies a predetermined condition, input time-series waveform data related to a fascia corresponding to each of the plurality of subjects to the trained machine learning model and acquire a known feature spectrum as a feature spectrum from output of a specific layer of the trained machine learning model for each of the plurality of pieces of time-series waveform data; a waveform acquisition unit configured to acquire target time-series waveform data, which is the time-series waveform data for each of different candidate loads, when a user executes a plurality of types of muscle strength training for which the different candidate loads are set; a second spectrum acquisition unit configured to input a plurality of pieces of the target time-series waveform data to the trained machine learning model and acquire a target feature spectrum as the feature spectrum from the output of the specific layer for each of the plurality of pieces of target time-series waveform data; and a specifying unit configured to calculate a spectrum similarity, which is a similarity between each of a plurality of the known feature spectra and each of a plurality of the target feature spectra, specify the spectrum similarity satisfying a predetermined extraction condition among a plurality of the calculated spectrum similarities, and specify the candidate load corresponding to the target time-series waveform data as a calculation source of the specified spectrum similarity. According to this aspect, the spectrum similarity satisfying the extraction condition is specified among the spectrum similarity between each of the plurality of known feature spectra and each of the plurality of target feature spectra for the different candidate loads, and the candidate load corresponding to the target time-series waveform data as a calculation source of the specified spectrum similarity is specified. Accordingly, the appropriate load can be specified for various users, and thus the specified appropriate load can be set as the load for the muscle strength training.

(7) According to a third aspect of the present disclosure, a non-transitory computer-readable storage medium storing a program causing a computer to execute load specification for a muscle strength training device is provided. The program includes: a function (a) of storing a vector neural network-based trained machine learning model having a plurality of vector neuron layers; a function (b) of, when a subject performs muscle strength training for which a reference load is set such that a muscle gain amount of the subject per unit time satisfies a predetermined condition, inputting time-series waveform data related to a fascia corresponding to each of a plurality of the subjects to the trained machine learning model and acquiring a known feature spectrum as a feature spectrum from output of a specific layer of the trained machine learning model for each of the plurality of pieces of time-series waveform data; a function (c) of acquiring target time-series waveform data, which is the time-series waveform data for each of different candidate loads, when a user executes a plurality of types of muscle strength training for which the different candidate loads are set; a function (d) of inputting a plurality of pieces of target time-series waveform data to the trained machine learning model and acquiring a target feature spectrum as the feature spectrum from the output of the specific layer for each of the plurality of pieces of target time-series waveform data; and a function (e) of calculating a spectrum similarity, which is a similarity between each of a plurality of the known feature spectra and each of a plurality of the target feature spectra, specifying the spectrum similarity satisfying a predetermined extraction condition among a plurality of the calculated spectrum similarities, and specifying the candidate load corresponding to the target time-series waveform data as a calculation source of the specified spectrum similarity. According to this aspect, the spectrum similarity satisfying the extraction condition is specified among the spectrum similarity between each of the plurality of known feature spectra and each of the plurality of target feature spectra for the different candidate loads, and the candidate load corresponding to the target time-series waveform data as a calculation source of the specified spectrum similarity is specified. Accordingly, the appropriate load can be specified for various users, and thus the specified appropriate load can be set as the load for the muscle strength training.

The present disclosure can be implemented in various forms other than the above. For example, the present disclosure can be implemented in the form of a non-transitory storage medium on which a computer program is recorded.

What is claimed is:

1. A load specifying method for a muscle strength training device, the method comprising:
a step (a) of preparing, for each of a plurality of subjects, time-series waveform data related to a fascia of the subject when the subject performs muscle strength training for which a reference load is set such that a muscle gain amount of the subject per unit time satisfies a predetermined condition;
a step (b) of inputting a plurality of pieces of the time-series waveform data to a vector neural network-based trained machine learning model having a plurality of vector neuron layers and acquiring a known feature spectrum as a feature spectrum from output of a specific layer of the trained machine learning model for each of the plurality of pieces of time-series waveform data;
a step (c) of acquiring target time-series waveform data, which is the time-series waveform data for each of different candidate loads, when a user executes a plurality of types of muscle strength training for which the different candidate loads are set;
a step (d) of inputting a plurality of pieces of the target time-series waveform data to the trained machine learning model and acquiring a target feature spectrum as the feature spectrum from the output of the specific layer for each of the plurality of pieces of target time-series waveform data; and
a step (e) of calculating a spectrum similarity, which is a similarity between each of a plurality of the known feature spectra and each of a plurality of the target feature spectra, specifying the spectrum similarity satisfying a predetermined extraction condition among a plurality of the calculated spectrum similarities, and specifying the candidate load corresponding to the target time-series waveform data as a calculation source of the specified spectrum similarity.

2. The load specifying method according to claim 1, wherein
in the step (a), the predetermined condition is a condition that the muscle gain amount per unit time is equal to or greater than a reference gain amount, and
in the step (e), the extraction condition is a condition that the spectrum similarity is equal to or greater than a threshold value.

3. The load specifying method according to claim 1, wherein
in the step (a), the predetermined condition is a condition that the muscle gain amount per unit time is maximum, and
in the step (e), the extraction condition is a condition that the spectrum similarity is highest among the plurality of spectrum similarities.

4. The load specifying method according to claim 1, wherein
the step (e) includes setting the specified candidate load as a load when the user performs the muscle strength training.

5. The load specifying method according to claim 1, wherein
in the step (c), the different candidate loads differ in magnitude of the loads at a certain interval.

6. A load specifying device for a muscle strength training device, the device comprising:
a storage device configured to store a vector neural network-based trained machine learning model having a plurality of vector neuron layers;
a first spectrum acquisition unit configured to, when a subject performs muscle strength training for which a reference load is set such that a muscle gain amount of the subject per unit time satisfies a predetermined condition, input time-series waveform data related to a fascia corresponding to each of a plurality of the subjects to the trained machine learning model and acquire a known feature spectrum as a feature spectrum from output of a specific layer of the trained machine learning model for each of the plurality of pieces of time-series waveform data;
a waveform acquisition unit configured to acquire target time-series waveform data, which is the time-series waveform data for each of different candidate loads, when a user executes a plurality of types of muscle strength training for which the different candidate loads are set;
a second spectrum acquisition unit configured to input a plurality of pieces of target time-series waveform data to the trained machine learning model and acquire a target feature spectrum as the feature spectrum from the output of the specific layer for each of the plurality of pieces of target time-series waveform data; and
a specifying unit configured to calculate a spectrum similarity, which is a similarity between each of a plurality of the known feature spectra and each of a plurality of the target feature spectra, specify the spectrum similarity satisfying a predetermined extraction condition among a plurality of the calculated spectrum similarities, and specify the candidate load corresponding to the target time-series waveform data as a calculation source of the specified spectrum similarity.

7. A non-transitory computer-readable storage medium storing a program causing a computer to execute load specification for a muscle strength training device, the program comprising:
a function (a) of storing a vector neural network-based trained machine learning model having a plurality of vector neuron layers;
a function (b) of, when a subject performs muscle strength training for which a reference load is set such that a muscle gain amount of the subject per unit time satisfies a predetermined condition, inputting time-series waveform data related to a fascia corresponding to each of a plurality of the subjects to the trained machine learning model and acquiring a known feature spectrum as a feature spectrum from output of a specific layer of the trained machine learning model for each of the plurality of pieces of time-series waveform data;
a function (c) of acquiring target time-series waveform data, which is the time-series waveform data for each of different candidate loads, when a user executes a plurality of types of muscle strength training for which the different candidate loads are set;
a function (d) of inputting a plurality of pieces of the target time-series waveform data to the trained machine learning model and acquiring a target feature spectrum as the feature spectrum from the output of the specific layer for each of the plurality of pieces of target time-series waveform data; and
a function (e) of calculating a spectrum similarity, which is a similarity between each of a plurality of the known feature spectra and each of a plurality of the target feature spectra, specifying the spectrum similarity satisfying a predetermined extraction condition among a plurality of the calculated spectrum similarities, and specifying the candidate load corresponding to the target time-series waveform data as a calculation source of the specified spectrum similarity.
